SEQUENCE LISTING (3 sequences)

SEQ ID NO: 1 - protein, 356 aa, Homo sapiens
MSPCGRARRQTSRGAMAVLAWKFPRTRLPMGASALCVVVLCWLYIFPVYRLPNEKEIVQGVLQQGTAWRRNQTAARAFRKQMEDCCDPAHLFAMTKMNSPMGKSMWYDGEFLYSFTIDNSTYSLFPQATPFQLPLKKCAVVGNGGILKKSGCGRQIDEANFVMRCNLPPLSSEYTKDVGAKSQLVTANPSIIRQRFQNLLWSRKTFVDNMKIYNHSYIYMPAFSMKTGTEPSLRVYYTLSDVGANQTVLFANPNFLRSIGKFWKSRGIHAKRLSTGLFLVSAALGLCEEVAIYGFWPFSVNMHEQPISHHYYDNVLPFSGFHAMPEEFLQLWYLHKIGALRMQLDPCEDTSLQPTS

SEQ ID NO: 2 - DNA, 21 bp, Artificial Sequence
  Description of Artificial SequenceAntisense oligonucleotide
cagtacagccatggcccctct

SEQ ID NO: 3 - DNA, 21 bp, Artificial Sequence
  Description of Artificial SequenceScramble oligonucleotide
cgacctacctatgcgctaccg

(19) United States
(12) Patent Application Publication
(10) Pub. No.: US 2004/0092438 A1
(43) Pub. Date: May 13, 2004

(54) Gd3-synthesis inhibitors for treating neuropathological disorders

(76) Inventors: Ana Martin-Villalba, Heidelberg (DE); Johannes Schenkel, Heidelberg (DE); Susanne Kleber, Heidelberg (DE); Roberto Testi, Roma (IT)

Correspondence Address: Keil & Weinkauf, 1101 Connecticut Avenue NW, Washington, DC, 20036, US

(21) Appl. No.: 10/169,989

(22) PCT Filed: Dec. 5, 2000
(86) PCT No.: PCT/EP00/12224

(30) Foreign Application Priority Data
Dec. 6, 1999 (DE) 19958684.5

(51) Int. Cl. A61K 38/17

(52) U.S. Cl. 514/12

(57) ABSTRACT

The present invention relates to the use of GD3 synthase inhibitors for treating neuropathological disorders, in particular cerebral ischemia, traumatic damage to the brain and spinal cord, and neurodegenerative disorders, and signs, symptoms and dysfunctions associated therewith, and to a method for the preparation of pharmaceuticals for the treatment of neuropathological disorders.

The present invention relates to the use of GD3 synthase inhibitors for treating neuropathological disorders, in particular cerebral ischemia, traumatic damage to the brain and spinal cord, and neurodegenerative disorders, and signs, symptoms and dysfunctions associated therewith and methods for producing pharmaceutical compositions for the treatment of neuropathological disorders which can be designed in particular as primary screening.

Sialic acids (Sia for short) play an important part in intercellular transmission processes, cytoplasmic interactions and cellular adhesion. They are found at the end of the hydrocarbon groups of glycoproteins and glycolipids, Sialic acids are introduced into these positions enzymatically as part of post-translational processes. Sia-α-2,3-Gal and Sia-α-2,8-Sia are sequence types frequently observed in gangliosides.

Enzymes which transfer sialic acids are glycosyltransferases and are referred to as sialyltransferases. In view of the large number of sialyloligosaccharide structures disclosed to date, it is assumed that at least 12 different sialyltransferases are involved in their synthesis.

Starting from an mRNA isolated from human melanoma cells, EP 0 654 529 describes an α-2,8-sialyltransferase whose physiological activity is useful for generating ganglioside GD3.

In EP 0 736 602, degenerate oligonucleotide primers directed at conserved sialyltransferase domains (sialyl motifs L and S) were used to subject the total mRNA from mouse brain to a PCR, leading to identification of another α-2,8-sialyltransferase, called ST8SiaIII therein. It is likewise a GD3 synthase, more accurately a Sia-α-2,3-Gal-β-1,4-GlcNAc-α-2,8-sialyltransferase from mouse brain. Use of the enzyme for sperm maturation, prevention of cancer metastases, inhibition of inflammatory processes and reactivation of nerve tissue is proposed.

Mention is also made of other α-2,8-sialyltransferases, specifically ST8SiaI (human, mouse) and ST8SiaII (STX) with N-glycan α-2,8-sialyltransferase activity.

Gangliosides are amphiphilic sialic acid-containing glycosphingolipids. They are ascribed with signal-transmitting properties. Quantitative and qualitative changes in gangliosides are observed during development, aging and disease of the central nervous system. In general, their concentration in the gray matter of the brain is higher than in the white and in peripheral nervous tissue. Neurons also usually show higher concentrations of gangliosides than astroglia. Gangliosides are mainly found in the plasma membrane and, in lower concentrations, on the endoplasmic reticulum, the Golgi apparatus, the lysosomes and the nuclear membrane. In the adult brain, the gangliosides GM1, GD1a, GD1b and GT1b account for 80-90% of the total ganglioside content, whereas GD3, a main component of the developing brain, is present only in traces. It is of interest that the GD3 level increases in pathological states, for example astrocytic scarring, Creutzfeld-Jakob and multiple sclerosis. In addition, there have been reports of a decrease in the total content of gangliosides in the brain in neurological disorders, for example early stages of Alzheimer's. Treatment of early stages of Alzheimer's and other neurodegenerative disorders such as parkinsonism, spinal cord trauma and at least transient stroke with GM1 has in turn proven beneficial.

Gangliosides are thought in this connection to be endogenous regenerating factors and are therefore the subject of research with a view to treatment of neurodegenerative disorders such as Alzheimer's and acute brain lesions such as cerebral ischemia, cf. Kracun, I. et al., Periodicum Biologorum, Vol. 97, No. 2, 113-118 (1995).

On the other hand, gangliosides are also described as cancer antigens. For example, EP 0 654 529 reports overexpression of GD3 in neuroectodermalen tumors for example malignant tumors. For this reason, GD3 is thought to be involved in the adhesion of cancer cells to extracellular substrates.

Kensuke Kawai et al. recently described in Psychiatry and Clinical Neuroscience, 53, 79-82 (1999), the detection of GD3 in the cytoplasm of reactive astrocytes (glia cells), specifically in brain sections taken at autopsy from people with Creutzfeld-Jakob or old cerebral infarcts. Astrocytes are thought to be actively involved in restoration of CNS lesions. This process, which is known as reactive glyosis, is ascribed with a regenerative function. The astrocytes proliferate as response to the neuronal cell loss. Accordingly, increases in the concentrations of GD3 and GM3 in astrocytes have been reported for the late phase of such pathological states, i.e. many hours or days after the onset of ischemic events and only during the course of neurodegenerative disorders.

Although gangliosides are generally regarded as neuroprotective, for example GM1 in models of stroke, Alzheimer's, parkinsonism etc., it has been reported by Testi et al. in Science, 227, 1652-1655 (1997), that cellular GD3 increases in lymphocytes at the start of CD95-induced apoptosis, and apoptosis can be prevented by inhibition of GD3 synthesis by means of antisense oligonucleotides. According to further investigations on hepatoma cells and isolated mitochondria, GD3 induces the opening of mitochondrial permeability transition pores and might be involved via this mechanism in cell death, cf. Scorrano L. et al., J. Biol. Chem., 274, 22581-22585 (1999) and Kristal, B. S. and Brown, A. M., J. Biol. Chem., 274, 23169-23175 (1999).

In contrast to the above results obtained on lymphocytes or hepatoma cells, it has been reported in other cases that, for example, there was a continuous decline in enzymic activity during an experimentally induced cerebral focal ischemia after occlusion of the middle cerebral artery (Geng Fuqiang et al., Acta Academiae Medicinae Shanghai, Vol. 22, No. 4, 295-298 (1995)).

CNS disorders currently affect large parts of the population. The numbers of patients are continually increasing in particular because of the increase in elderly people. Neuropathological states such as cerebral ischemia, stroke, and neurodegenerative disorders, for example dementia, especially Alzheimer's dementia, demyelinating disorders, especially multiple sclerosis, and brain tumors lead to damage to the brain and the neuronal deficits associated therewith.

Great efforts are being made, especially with a view to treating stroke as the commonest life-threatening neurological disorder, to ensure that an affected patient is admitted as quickly as possible to hospital. However, the therapeutic possibilities are limited, irrespective of the partial successes achieved by rt-PA and aspirin treatment.

It is an object of the present invention to provide novel applications of a modulation of GD3 synthase activity.

We have found that this object is achieved in that inhibition of GD3 synthase activity makes it possible to treat neuropathological disorders and the signs, symptoms and dysfunctions associated therewith.

The present invention therefore relates to the use of at least one GD3 synthase inhibitor for treating neuropathological disorders and the signs, symptoms and dysfunctions associated therewith.

Neuropathological disorders mean according to the invention disorders accompanied by neurological deficits, i.e. a state characterized by disturbances of neurological functions.

This state may exist transiently, progressively or persistently.

It is preferred according to the invention to treat cerebral ischemia, traumatic brain and spinal cord damage, that is to say, in particular, brain and spinal cord trauma, and neurodegenerative disorders, especially dementia, in particular Alzheimer's dementia, parkinsonism, ALS (amyotrophic lateral sclerosis), multiple sclerosis.

It is particularly preferred to treat neuropathological disorders associated with cerebral ischemia and, in particular, those attributable thereto. Mention should be made here in particular of stroke (Synonym: cerebral apoplexy, cerebral or apoplectic insult, cerebrovascular accident). It is also possible to treat according to the invention transient ischemic attacks, reversible ischemic neurological deficits, prolonged reversible ischemic neurological deficits, partially reversible ischemic neurological symptoms and also persistent complete cerebral infarcts. The treatment of acute forms is particularly advantageous according to the invention.

The forms of neuropathological disorders preferably treated according to the invention are based on one or more of the nerve tissue lesions listed hereinafter: degeneration or death of neurons, in particular of gangliocytes, for example chromatolysis, nuclear membrane blurring, plasmolysis, cytoplasmic vacuolation and encrustation, cerebral parenchymal necroses, cerebral edemas, neuronal changes caused by oxygen deficiency, atrophy, morphological changes such as demyelination, in particular myelinic degeneration, perivascular infiltrates, glial proliferation and/or glial scars; degeneration of the substantia nigra.

The indication to be treated according to the invention is frequently characterized by a progressive development, i.e. the states described above change with time, the severity usually increasing and, where appropriate, states possibly inter-converting or existing states being joined by other states.

The treatment according to the invention of neuropathological disorders or of the states underlying them permits treatment of a number of other signs, symptoms and/or dysfunctions associated with the neuropathological disorders, i.e. in particular accompanying the pathological states described above. These include, for example, shock lung; cranial nerve deficits, for example retrobulbar neuritis, ophthal moplegias, staccato speech, spastic paralyses, cerebellar symptoms, sensitivity disorders, bladder and rectal disorders, euphoria, dementia, hypokinesia and akinesia, absence of synkinesis, small-step gait, bent posture of trunk and limbs, pro-, retro- and lateropulsion, tremor, mask-like face, monotonous speech, depression, apathy, labile or rigid affectivity, deficient spontaneity and irresolution, slowing of thought, reduced association ability; muscle atrophy.

Treatment in the sense according to the invention, comprises not only the treatment of acute or chronic signs, symptoms and/or dysfunctions, but also prophylactic treatment (prevention). One purpose of the acute or chronic treatment is to remedy the disorders, to regulate the states, or to alleviate the signs, symptoms and/or dysfunctions. Under a particular aspect it is the purpose of the treatment to diminish the enzymatic activity connected with GD3 synthase. A purpose of the prophylactic (preventive) treatment is to inhibit the occurrence of the disorders, states, signs, symptoms and/or dysfunctions or to delay their occurrence. The treatment may be directed to the symptoms, for example as a symptom suppression. It may take place as a short-term or a medium-term measure or it may also be a long-term treatment, e. g. within the framework of a maintenance therapy.

The term “GD3 synthase inhibitor” describes substances which inhibit the enzymatic activity of GD3 synthases or expression thereof. Enzyme inhibition means in this connection a reduction in the enzyme activity, especially of the activity as glycosyltransferase, sialyltransferase, α-2,8-sialyltransferase and, in particular, Sia-α-2,3-Gal-β-1,4-GlcNAc-α-2,8-sialyltransferase (EC 2.4.99.8). The GD3 synthase activity results, for example, in the reaction of a glycosyl donor with a glycosyl acceptor to give an oligosaccharide product. This usually entails a monosaccharide being transferred from the glycosyl donor to the glycosyl acceptor. The glycosyl donor is ordinarily a nucleotide linked via its phosphate groups to a monosaccharide. Thus, for example, glucose (Glc), galactose (Gal), N-acetylglucosamine (GlcNAc), N-acetylgalactosamine (GalNAC), xylose (Xyl) and glucuronic acid (GlcA) are transferred by uridine diphosphate donors, fucose (Fuc) and mannose (Man) are transferred by guanosine diphosphate donors and sialic acid (N-acetylneuraminic acid, NeuAc, NANA) is transferred by cytidine monophosphate donors. The preferred glycosyl donor for the sialyltransferase activity is CMP-NeuAc. The glycosyl acceptors are usually glycoproteins, oligosaccharides or glycolipids. The sialyltransferase activity of the GD3 synthase is directed in particular at glycosyl acceptors having a terminal β-Gal or sialic acid residue. The α-2,8-sialyltransferase activity of the GD3 synthase links a sialic acid residue via an α-2,8-linkage to a terminal sialic acid residue. It is preferred according to the invention to inhibit the activity of the GD3 synthase with which a sialic acid residue is transferred to the sequence Sia-α-2,3-Gal-β-1,4-GlcNAc. It is very particularly preferred to transfer a sialic acid residue to the ganglioside GM3 to form an α-2,8 linkage, namely conversion of GM3 into GD3. (GD3=II³(NeuAc-α-2,8-NeuAc)LacCer).

It is preferred according to the invention to inhibit mammalian GD3 synthase and, in particular, human GD3 synthase, in particular the GD3 synthase having the amino acid sequence SEQ ID NO:1, and homologous sequences thereof. These are generally transmembrane proteins of type II (about 40 kD) which are localized in the early Golgi and whose catalytic domain is usually characterized by COOH-terminal sialyl motif S.

Inhibition of GD3 synthase leads to diminishing the enzymatic activity connected with GD3 synthase. This may happen via the modulation of the enzyme itself or the expressed amount of the enzyme. According to a special aspect of the present invention, the modulation particularly relates to the neuronal tissue and above all the neurons themselves.

Inhibitors according to the invention usually bind to GD3 synthases or GD3-synthase-encoding nucleic acids, for example DNA or mRNA. Binding means any molecular interaction between inhibitor and enzyme or nucleic acid, in particular under physiological conditions. These are usually classical interactions, which include electrostatic forces, van der Waals forces, hydrogen bonds, hydrophobic links, or metal complex-like coordinate links. In addition to the aforementioned reversible molecular interactions, it is also possible to consider irreversible interactions between inhibitor and enzyme, for example covalent bonds.

Enzyme inhibitors usually bind in the region of the or of one of the active domains of GD3 synthase and compete with other substrates for their binding site (competition). Accordingly, competitive enzyme inhibitors mean those which compete with a comparison substrate, in the present case glycosyl donors and/or glycosyl acceptors, preferably UDP-NeuAc and/or GM3, for binding to GD3 synthases, i.e. the binding of one impedes the binding of the other. Because of this binding to GD3 synthase it is also possible to refer to competitive enzyme inhibitors as GD3 synthase substrates. These inhibitors are preferably substrates which, by comparison with the natural substrate(s) are inaccessible or at least less accessible to the catalytic activity of GD3 synthase, i.e. they are converted by GD3 synthase to a comparatively small extent or not at all. It is likewise possible to use noncompetitive inhibitors which, for example, bind essentially irreversibly to active domains, or elsewhere on the GD3 synthase and, for example, have an influence on the enzyme activity through allosteric effects.

The principle applying at least in the case of competitive inhibition is that the displacement of a substrate by an inhibitor increases with decreasing binding affinity of the competing substrate or increasing binding affinity of the inhibitor. It is therefore expedient for inhibitors which can be used according to the invention to have a high binding affinity for GD3 synthase. Such a favorable binding affinity allows naturally occurring enzyme substrates, i.e. in particular glycosyl donors and/or glycosyl acceptors, for example UDP-NeuAc and/or GM3, to be effectively displaced, there being a decrease in the inhibitor concentration necessary for binding a particular amount of this inhibitor to the enzyme or for displacing a particular amount of a substrate as the binding affinity of the inhibitor increases. In relation to medical use, therefore, preferred inhibitors have a binding affinity which is so large that they can be administered as active ingredient in acceptable amounts as part of an effective medical treatment. Inhibitors according to the invention are therefore preferably administered in daily doses of about 0.01 to 30 mg/kg of bodyweight and, in particular, of about 0.1 to 15 mg/kg of bodyweight.

One possible way of expressing the binding affinity is provided by the competition experiments mentioned above, in which there is measurement of the inhibitor concentration which inhibits the enzyme by 50% in relation to the conversion of another substrate (IC₅₀ values). Thus, it is also possible to analyze the competitive inhibition of the binding of GD3 synthase inhibitors to the effect that inhibitors preferred according to the invention have half-maximal inhibition constants IC₅₀ in vitro of less than 10⁻⁴ M, preferably of less than 10⁻⁵ M and, in particular, of less than 10⁻⁶ M.

The expression inhibitors are substances in the widest sense which quantitatively or qualitatively impair the synthesis of GD3 synthase, in particular oligonucleotides or suitable synthetic compounds which act, for example, in the manner of an antisense RNA or DNA, or in the manner of the triple helix technique.

It is known, for example, that the gangliosides GQlb, GTla and GTlb inhibit GD3 synthase activity.

It is also known that glycosylation inhibitors such as tunicamycin impair the glycosylation of GD3 synthase, whereby the latter may lose its enzymatic activity.

GD3 synthase-specific antibodies may also be useful as GD3 synthase inhibitors. These may be polyclonal antisera, monoclonal antibodies, antibody fragments such as F(ab), Fc, etc., chimeric, humanised and recombinant antibodies. Such antibodies can be produced in a manner known per se. It is possible to use as immunogen GD3 synthase as such or antigenic fragments thereof usually coupled to conventional carrier proteins.

Low molecular weight GD3 synthase inhibitors, usually synthetic compounds, are advantageously useful in many respects.

It is also possible to use aptamers, which are nucleic acids, usually oligonucleotides, with sufficient affinity for GD3 synthase, as inhibitors.

The use according to the invention is not restricted to the aforementioned inhibitors. On the contrary, it is possible to use according to the invention as GD3 synthase inhibitor any substance in whose presence the GD3 synthase activity is less than in its absence.

For measuring GD3 synthase activity there are known test systems in which GD3 synthase inhibitors which can be used according to the invention lead, as test substance, to a reduction in the GD3 synthase activity. These test systems are usually based on the conversion of labeled substrates, it being possible for donor and/or acceptor substrate to be labeled, and thus, for example, on the incorporation of labeled donor substrates, especially fluorescence-, for example fluorescein-labeled, and radiolabeled, for example ³⁵S-, ¹⁴C-, or ³H-labeled substrates, e.g. CMP-[¹⁴C]Neu5Ac (synonym: [¹⁴C]-labeled CMP-β-D-sialic acid, CMP-NANA or CMP-N-acetylneuraminic acid; cf. Yusuf et al., Eur. J. Biochem. 134(1):47-54 (1983)), into acceptor substrates, usually glycoproteins, oligosaccharides or glycolipids, especially those with the sequence Gal-β-1,4-GlcNAc and, in particular, gangliosides, e.g. GM3, NeuAc-α-2,3-Gal-β-1,4-GlcNAc, it being possible to follow the conversion, i.e. the attachment of this substrate to an acceptor substrate and the assembly associated therewith, of particular glycoproteins or glycolipids by means of the label. It is also possible for the acceptor substrates to be labeled. The reaction products can be fractionated by chromatography, for example by thin-layer chromatography, HPLC or by electrophoresis. Glycoproteins in the reaction mixture can be fractionated by electrophoresis, e.g. by SDS-PAGE, and stained, e.g. with Coomassie Brilliant Blue. Glycolipids in the reaction mixture can be fractionated by HPLC and stained, e.g. with orcinol/sulfuric acid. It is thus possible to quantify the incorporation of the label into particular glycoproteins or glycolipids.

If the inhibition affects the expression of GD3 synthase, it is possible to detect the nature and amount of the expressed GD3 synthase immunologiscally, for example using the antibody R24 which is directed against GD3 synthase, or using antibodies against signal or carrier peptides which are coexpressed where appropriate, for example influenza virus hemagglutinin nonapeptide, or via the incorporation of labels in GD3 synthase, for example [2-³H]-labeled mannose.

The substrates can be obtained by biological means or be synthesized by chemical means. CMP-5-fluoresceinyl-sialic acid and [¹⁴C]-labeled CMP-sialic acid are commercially available (e.g. Calbiochem and Amersham respectively). The same applies to GM3 and pyrene-labeled GM3 (e.g. Sigma). It may be advantageous to couple such substrates to a solid carrier, which facilitates the detection of liberated fragments. The coupling chemistry usual in this area can be used for this purpose.

The GD3 synthase used for tests can be of natural or recombinant origin. For example, it is possible to isolate Golgi vesicles, for example from rat livers, and to determine the GD3 synthase activity in the homogenates obtained therefrom. It can also be produced recombinantly using the expression systems described in WO 94/23020 or by Martina et al. in J. Biol. Chem. 273(6):3725-3731 (1998).

The present invention therefore also relates to a method for identifying GD3 synthase inhibitors, wherein the activity of GD3 synthase is determined in the presence and in the absence of at least one test substance.

The test systems which are described above and others which are suitable in a similar way can form the basis for in vitro screening methods, preferably for primary screening, with which it is possible to select from a large number of different substances those useful in terms of the application according to the invention. Thus, the present invention also relates to corresponding methods for identifying the active substances for the treatment of neuropathological disorders and based upon this the preparation of pharmaceutical compositions for the treatment of neuropathological disorders. Such method for the development of active substances for the treatment of neuropathological disorders is characterized in that at first GD3 synthase inhibitors are selected from a variety of substances and that these are used in the preparation of the pharmaceutical composition. For example, it is possible to set up by means of combinatorial chemistry wide-ranging substance banks which comprise myriads of potential active substances. The screening of combinatorial substance libraries for substances with a desired activity can be automated. Screening robots are used for efficient analysis of the individual assays which are preferably arranged on microtiter plates.

A particularly efficient technology for carrying out such methods is the scintillation proximity assay, abbreviated to SPA, which is well known in the area of screening for active substances. Kits and components for carrying out this assay can be purchased commercially, for example from Amersham Pharmacia Biotech. For enzymatic test applications, in principle solubilized or membrane-bound substrates are immobilized on small, scintillant-containing fluomicrospheres. Depending on the nature of the enzymatic activity to be tested, the substrate is radiolabeled and the scintillant is stimulated to emit light for as long as the scintillant and radiolabel are close in space, or the radiolabel is introduced into the immobilized substrate precisely by the enzymic activity to be measured, and consequently stimulates the scintillant to emit light. The resulting test formats involve measurement respectively of a decreasing or increasing signal intensity.

Another particularly efficient technology for carrying out such methods is the flash plate technology well known in the area of screening for active substances. Kits and components for carrying out this assay can be purchased commercially, for example from NEN Life Science Products. This principle is likewise based on microtiter plates (96- or 384-well) coated with scintillant.

Other test methods suitable in particular for secondary screening are based on in vitro and in vivo models of indications to be treated according to the invention.

For example, a number of in vitro and in vivo models of stroke are known and established. Thus, neurons can be cultivated with oxygen/glucose deficiency and the proportion of dead cells can be determined. Inhibitors which can be used according to the invention reduce the proportion of dead cells.

One in vivo possibility is to induce an ischemia in experimental animals by, for example, clamping off the middle cerebral artery (focal cerebral ischemia). The extent of ischemia induced can be determined, for example, histologically. Inhibitors which can be used according to the invention reduce the extent of the ischemia induced.

The use according to the invention of GD3 synthase inhibitors comprises a method for the purpose of treatment. This entails administration of an effective amount of one or more GD3 synthase inhibitors, usually formulated in accordance with pharmaceutical and veterinary medical practice, to the individual to be treated, preferably a mammal, preferably a human, agricultural or domestic animal. Whether such a treatment is indicated and what form it should take depend on the individual case and are subject to medical assessment (diagnosis) taking account of the signs, symptoms and/or dysfunctions which are present and the risks of developing particular signs, symptoms and/or dysfunctions, and other factors.

The treatment usually takes place by a single or multiple daily administration where appropriate together or in alternation with other active substances or products containing active substances, so that a daily dose of about 0.001 g to 10 g, preferably of about 0.001 g to about 1 g, is given to an individual to be treated.

The invention also relates to the production of pharmaceutical compositions for the treatment of an individual, preferably a mammal, in particular a human, agricultural or domestic animal. Thus, the inhibitors are ordinarily administered in the form of pharmaceutical compositions which comprise a pharmaceutically suitable excipient with at least one inhibitor according to the invention and, where appropriate, other active substances. These compositions can be administered orally, rectally, transdermally, subcutaneously, intravenously, intramuscularly or intranasally, for example.

Examples of suitable pharmaceutical formulations are solid drug forms such as oral powders, dusting powders, granules, tablets, pastilles, sachets, cachets, coated tablets, capsules such as hard and soft gelatin capsules, suppositories or vaginal drug forms, semisolid drug forms such as ointments, creams, hydrogels, pastes or plasters, and liquid drug forms such as solutions, emulsions, in particular oil-in-water emulsions, suspensions, for example lotions, preparations for injection and infusion, eye drops and ear drops. It is also possible to use implanted delivery devices for administering inhibitors according to the invention. Application of liposomes, microspheres or polymer matrices is also possible.

To produce the compositions, inhibitors according to the invention are ordinarily mixed or diluted with an excipient. Excipients may be solid, semisolid or liquid materials which serve as vehicle, carrier or medium for the active substance.

Examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methylcellulose. The formulations may also comprise pharmaceutically acceptable carriers or conventional ancillary substances such as lubricants, for example talc, magnesium stearate and mineral oil; wetting agents; emulsifying and suspending agents; preserving agents such as methyl and propyl hydroxybenzoates; antioxidants; anti-irritants; chelating agents; tablet coating aids; emulsion stabilizers; film formers; gel formers; odor-masking agents; masking flavors; resins; hydrocolloids; solvents; solubilizers; neutralizing agents; permeation accelerants; pigments, quaternary ammonium compounds; refatting and superfatting agents; ointment, cream or oil bases; silicone derivatives; spreading aids; stabilizers; sterilants; suppositories bases; tablet ancillary substances such as binders, fillers, lubricants, disintegrants or coatings; propellants; desiccants; opacifiers; thickeners; waxes; plasticizers; white oils. Arrangement thereof depends on expert knowledge as presented, for example, in Fiedler, H. P., Lexikon der Hilfsstoffe für Pharmazie, Kosmetik und angrenzende Gebiete, 4th edition, Aulendorf: ECV-Editio-Cantor-Verlag, 1996.

The present invention is explained in detail by means of the following examples without being restricted thereto.

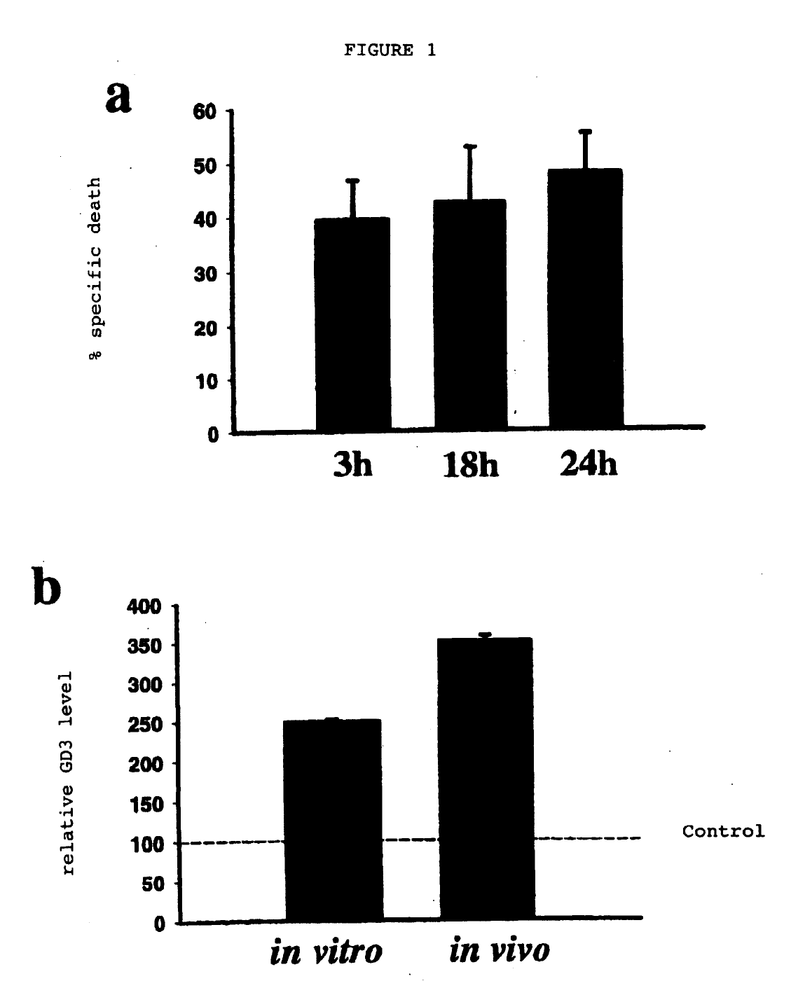

FIG. 1a shows the percentage of primary neurons which have died specifically after a 6-hour oxygen/glucose deficiency and reperfusion times of 3 h, 18 h and 24 h;

FIG. 1b shows changes in GD3 levels in neurons after 24-hour reperfusion (in vitro) and in neurons in the ischemic hemisphere after a 90-minute MCA occlusion and subsequent 24-hour reperfusion (in vivo) (% GD3 level relative to 100% control; average±standard deviation; n=3);

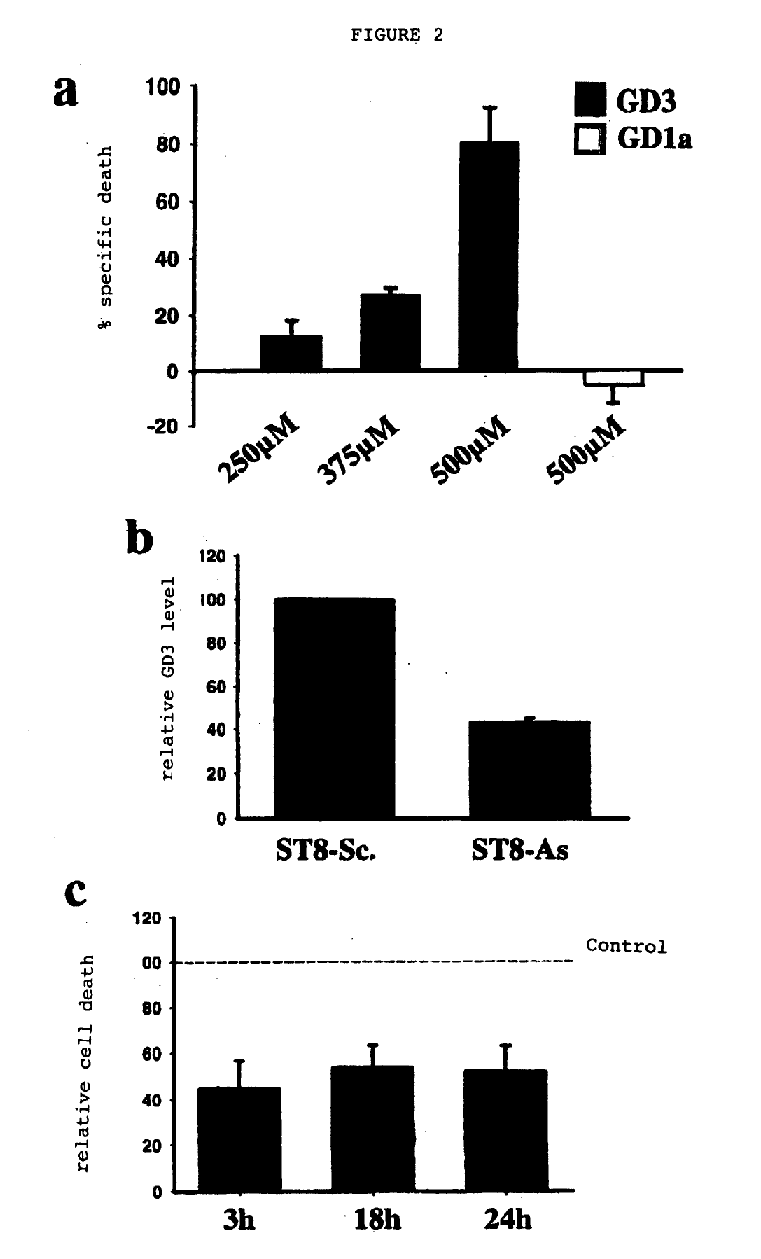

FIG. 2a shows the percentage increase in the specific death of primary neurons 24 h after addition of GD3 in final concentrations of 250 μM, 375 μM and 500 μM and of GD1a in a final concentration of 500 μM, to the culture medium;

FIG. 2b shows the GD3 levels induced by a 6-hour oxygen/glucose deficiency and after 24-hour reperfusion, after addition of antisense oligodeoxynucleotides against GD3 synthase (ST8-As) or oligodeoxynucleotides with a randomly arranged sequence of the same nucleotides (ST8-Sc) 48 h before induction of the oxygen/glucose deficiency (% GD3 levels of ST8-As-treated neurons relative to 100% GD3 level of ST8-Sc-treated neurons; n=3);

FIG. 2c shows the specific death of primary neurons after a 6-hour oxygen/glucose deficiency and subsequent 3-hour, 18-hour and 24-hour reperfusion after addition of antisense oligodeoxynucleotides against GD3 synthase (ST8-As) or oligodeoxynucleotides with a randomly arranged sequence of the same nucleotides (ST8-Sc) to the culture medium 48 h before induction of the oxygen/glucose deficiency (% death of ST8-AS-treated neurons relative to 100% death of ST8-Sc-treated neurons, n=5);

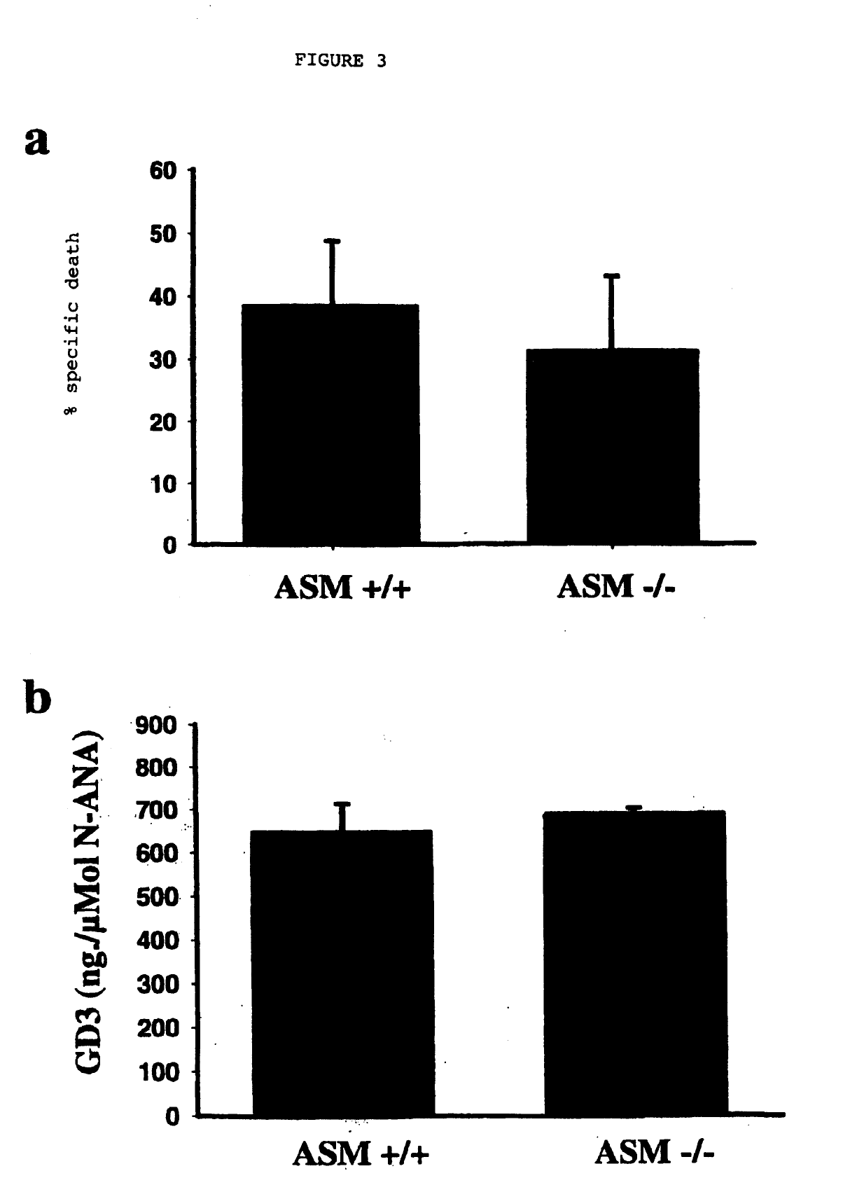

FIG. 3a shows the specific death of primary neurons from mice with acid sphingomyelinase deficiency (ASM^{-/}⁻) and from wild-type mice of the same generation (ASM^{+/}⁺) after a 6-hour oxygen/glucose deficiency and subsequent 24-hour reperfusion;

FIG. 3b shows the GD3-concentration in neurons from mice with acid sphingomyelinase deficiency (ASM^{-/}⁻) and from wild-type mice of the same generation (ASM^{+/}⁺) after a 6-hour oxygen/glucose deficiency and 24-hour reperfusion (ng/μmol of neorominic acid; average±standard deviation; n=3);

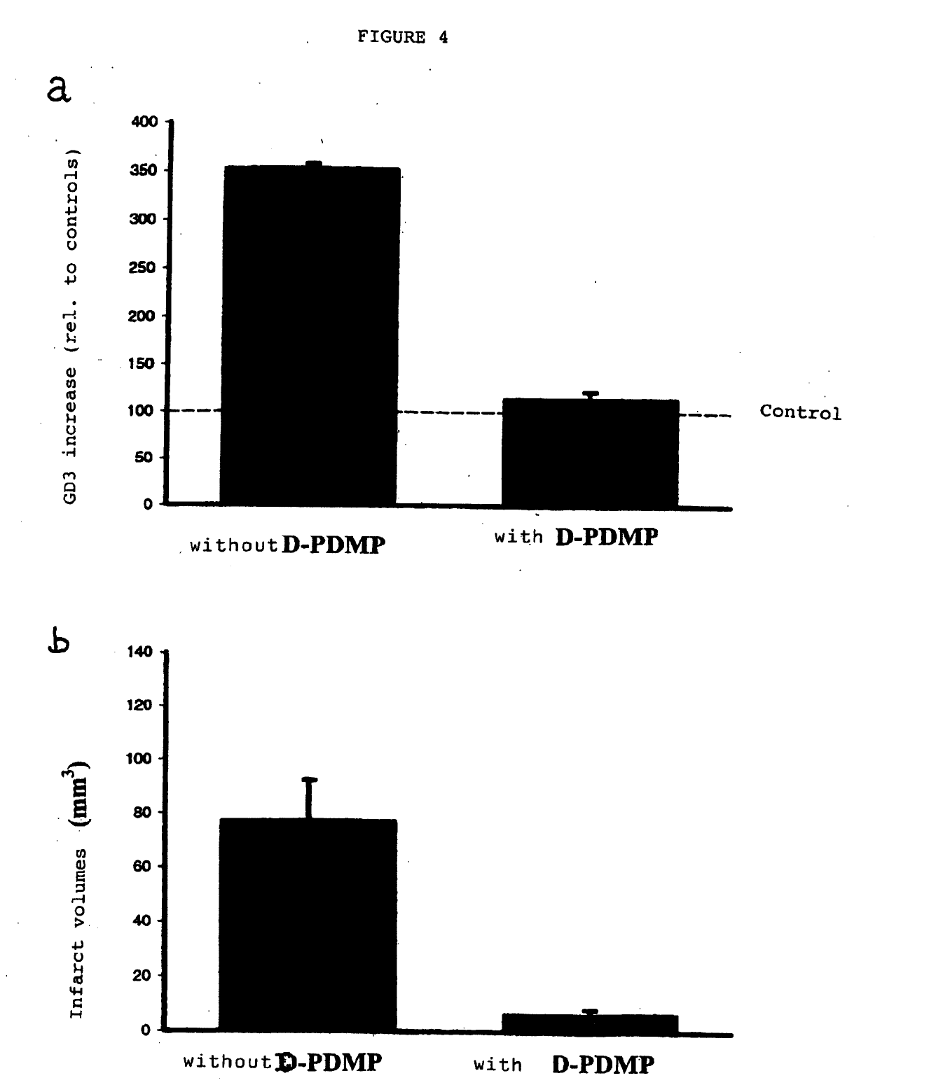

FIG. 4a shows the GD3 levels in the ischemic hemisphere of mice after a 90-minute MCA occlusion and 24-hour reperfusion without previous administration of D-PDMP (without D-PDMP) or after previous administration of D-PDMP (with D-PDMP) (% GD3 levels relative to 100% GD3 levels in the nonischemic hemisphere; n=3);

FIG. 4b shows the infarct volumes determined 24 h after a 90-minute MCA occlusion in untreated (without D-PDMP, n=7) and treated (with D-PDMP, n=6) mice.

### REFERENCE EXAMPLE 1

### Animal Model of Ischemia

A focal cerebral ischemia was induced in mice (C57BL6 strain) by occlusion of the middle cerebral artery (MCA) (Longa et al, Stroke 20, 84-91 (1989)). This was done by pushing a siliconized nylon suture through the common carotid artery as far as the MCA and stopping the blood flow through the MCA. After 90 min, the thread was withdrawn and thus the blood circulation was restored. The animals were under deep anesthesia (ketamine and Rompun; each 150 mg/kg of bodyweight) and the rectal temperature was maintained at about 37° C. with heat lamps and a heated cushion throughout the operation. After an observation period of 24 hours, the animals were again anesthetized, and the brains were dissected after perfusion.

### REFERENCE EXAMPLE 2

### Determination of the Infarct Volume

Cryostatic coronal sections were prepared from the forebrains obtained in Reference Example 1, with a thickness of 20 μm and with a spacing of 400 μm, and subjected to a silver stain. This was done by impregnating the sections with a silver nitrate/lithium carbonate solution for 2 min and developing with a hydroquinone/formaldehyde solution for 3 min (Vogel, R. A., Clin. Cardiol., II 34-9 (1999)). The stained sections were scanned directly (MCID-M4,3.0; Imaging Res. Inc.). The infarct volume was determined by numerical integration by means of digital planimetry (Swanson, R. A. et al, Stroke 21, 322-7 (1990); Lin, T. N., et al., Stroke 24, 117-21 (1993)) of the scanned regions with considerable paling (corrected for cerebral odema×section thickness). All the results are stated as average ±standard deviation. The significance was determined by the Mann-Whitney test. Maps of the frequency distribution of infarcts were constructed (Belayev, L. et al., Neuroreport 8, 55-9 (1996); Schneider, A. et al., Biochemistry 38, 3549-58 (1999)) by scanning each of the sections in a series, and localizing the infarcts and projecting them onto a mask. Averaging took place with the Scion-Image b 3.b.

### REFERENCE EXAMPLE 3

### Immunohistochemistry

Coronal cryostatic sections (20 μm) from Reference Example 2 were incubated either with the monoclonal anti-GD3-IgG3 mouse antibody (R24) or with the polyclonal antibodies anti-NF200 (Sigma, Deisenhofen, Germany), anti-GFAP (Chemicon, Canada) or anti-cytochrome c (H-104; Santa Cruz, Canada) for 48-72 h. The immunoreactivities were visualized either using a monoclonal or polyclonal Cy3/FITC-labeled second antibody (Dianova, Hamburg, Germany) or using the conventional avidin-biotin complexation method (Vectastain, Vector Lab, U.S.A.).

### REFERENCE EXAMPLE 4

### Transferase dUTPnick End-labeling (TUNEL)

Cryostatic frontal sections (25 μm) were treated by the transferase dUTPnick end-labeling technique (Gavrieli, Y. et al., J. Cell Biol. 119, 493-501 (1992)). Cell nuclei were detached from proteins by incubation of PBS with 1% Triton (PBST) at 4° C. overnight. Endogenous peroxidases were inactivated by covering the sections with 2% H₂O₂ at room temperature for 5 min. These sections were incubated with 0.3 μl of Flu-dUTP (Amersham, Braunschweig, Germany), 1 μl of terminal deoxynucleotidyltransferase (TdT), 10 μl of 5x TdT buffer, 2 μl of CoCl₂ (Boehringer, Mannheim, Germany), 0.3 μl of dATP (Perkin Elmer) and 36.4 μl of distilled H₂O in a humidity chamber at 37° C. for 90 min. The reaction was stopped by transferring the slides into a TE buffer at room temperature for 10 min. Normal cell nuclei, which contained only insignificant amounts of DNA 3′-OH ends, showed no staining with this technique. Cells with necrotic morphology and detectable concentrations of DNA ends showed more diffuse labeling than apoptotic cell nuclei. Control sections were incubated either without enzyme or without nucleotide.

### REFERENCE EXAMPLE 5

### Cell Culture

Neurons were obtained from transgenic mice whose acid sphingomyelinase had been inactivated (ASM^{-/-}; Hirunouchi, K., et al., Nature Genetics 10, 288-294 (1995)) and wild-type mice of the same generation (ASM^{+/+}). Primary neuron cultures were prepared from 15- to 17-day mouse fetuses as previously described by Dawson, T. M., et al, Ann. neurol. 30, 843-6 (1991). In accordance with this, trituration in MEM medium with 20% horse serum, 25 mM glucose and 2 mM L-glutamine (all from Gibco/Life Technologies, Paisley, Scotland) and subsequent 30-minute digestion in 0.025 trypsin/sodium chloride solution resulted in cortical neurons. The cells were applied to polyornithine-coated 24-well plates (Sigma, Deisenhofen, Germany). After 4 days, the cells were treated with cytosine arabinoside (5 μM) for a further four days in order to inhibit the proliferation of non-neural cells. The cell cultures were then kept at 8% CO₂ and 37° C. in MEM, 10% horse serum, 25 mM glucose and 2 mM L-glutamine in a humidity incubator. The neurons were allowed to mature in culture for at least 8 days before being used for the experiment. The proportion of glial cells in the culture was determined using an antibody directed against glial fibrillary acidic protein (GFAP) to be less than 10%.

### REFERENCE EXAMPLE 6

### Oxygen/glucose Deficiency in Vitro

The procedure for generating a combined oxygen/glucose deficiency was known per se (Kaku, D. A., et al., Brain Res. 554:344-7; Monyer, H. et al., Neuron 8, 967-73 (1992); with slight modifications). The culture medium was replaced by MEM, 1% horse serum and 2 mM L-glutamine. The cultures were kept in an anaerobic chamber which contained a gas mixture comprising 5% H₂, 85% N₂ and 5% CO₂, at 37° C. and 100% humidity for 6 h. The oxygen/glucose deficiency was abolished by removing the cultures from the chamber and adding horse serum and glucose to a final concentration of 10% and 25 mM respectively. The cultures were then replaced in a humidity incubator containing 8% CO₂ and atmospheric oxygen for a further 3 h, 18 h or 24 h. To inhibit GD3 synthesis, the cultures were incubated with 80 nM of GD3 synthase antisense oligodeoxynucleotides (SEQ ID NO:2) and a randomly assembled oligodeoxynucleotide (SEQ ID NO:3) 48 h before induction of the oxygen/glucose deficiency. Bovine brain GD3 was purchased from Sigma (Deisenhofen, Germany).

The percentage of dead cells was determined by trypan blue exclusion and expressed as percentage of the specific death. This was calculated as follows:

% specific death=(detected death−spontaneous death)/(100−spontaneous death)×100

### REFERENCE EXAMPLE 7

### Thin-layer Chromatography (GD3 analysis)

GD3 was determined as previously described by De Maria, R., et al., Science 277, 1652-5 (1997). Thus, tissues and cells were disrupted by two freeze-thaw cycles. The polar lipids were extracted, and the gangliosides were chromatographed on silica gel plates 60 for high performance thin-layer chromatography (HPTLC) (Merck, Darmstadt, Germany), dried and visualized with resorcinol. Identical amounts of neuraminic acid were found with the thiobarbituric acid assay (Warren, L., J. Biol. Chem. 234, 1871-1975 (1959)). For the immunostaining, the plates were coated with 0.5% poly(isobutyl methacrylate) in hexane, dried, incubated with the monoclonal antibody R24 (IgG3) directed against GD3 for 1 h, and read via the immunoperoxidase stain. The bands were then scanned and analyzed by densitometry using the AIDA-1000/1B image analyzer (Raytest Isotopenmessgerate, Straubenhardt, Germany).

### EXAMPLE 1

### GD3 Levels in Neurons After Induced Ischemia

A 6-hour oxygen/glucose deficiency and increasing reperfusion times as described in Reference Example 6 induced specific death in up to 48% of primary neurons (FIG. 1a). It was possible to show by quantitative thin-layer chromatography as described in Reference Example 7 that this increase in neuronal death was accompanied by a rise in the GD3 levels (149.45±3.00% compared with the untreated controls, n=3; FIG. 1b). The same mode of analysis revealed for the ischemic hemisphere of mice treated as described in Reference Example 1 a 250% rise in the GD3 levels compared with the non-ischemic hemisphere (252.03±4.81%, n=3; FIG. 1b).

It was possible to examine the distribution of GD3 accumulation within the ischemic brain by immunohistochemistry. GD3 was detected in the lateral stratum and the adjacent neocortex only 4 h after MCA occlusion. The GD3 accumulation increased progressively up to 24 h after ischemia; at this time, GD3 was detected in the lateral stratum, ventrolateral thalamus, the CA1 and CA3 hippocampus layers and the cortex adjoining the ischemic lesion. GD3 was undetectable either in the brains of sham-operated animals or in the control stains carried out without the first antibody.

Double-labeling investigations showed that GD3 neosynthesis takes place mainly in neurons after a 24-hour reperfusion. In addition, increasing cellular amounts of GD3 correlated with an enhanced mitochondrial release of cytochrome c. Accordingly, GD3 was found in apoptotic neurons.

### EXAMPLE 2

GD3 Promotes Neuronal Death

Cortical neuronal cells were treated with increasing doses of GD3. The specific neuronal death increased as a function of the dose, specifically up to 80% 24 h after the addition of 500 μM GD3 (FIG. 2a). Another disialoganglioside GDla showed no effect at the highest dose used (FIG. 2a).

Addition of GD3 synthase antisense oligodeoxynucleotides, but not addition of a randomly assembled sequence of the same nucleotides, 48 h before induction of the oxygen/glucose deficiency with a subsequent 24-hour reperfusion as described in Reference Example 6 led to a reduction in the accumulation of GD3 to 55% of the controls (FIG. 2b). This reduction led to a decrease in the mortality in antisense-treated cultures to 50% of the level found in the cultures which had been treated only with the randomly arranged sequence of the nucleotides in the antisense oligodeoxynucleotide sequence (FIG. 2c).

### EXAMPLE 3

### No Dependence on the Acid Sphingomyelinase Activity

No significant difference was detectable between ASM^{-/-} and ASM^{+/+} mice in respect of the specific neuronal death assessed as described in Reference Example 6 (31.09±11.95 vs. 38.28±10.28% specific death; FIG. 3a). Accordingly, GD3 accumulated in approximately identical amounts after oxygen/glucose deficiency/reperfusion in both the ASM^{-/-} and ASM^{+/+} neurons (689.43±13.53 vs. 647.53±67.21 mg of GD3/μmol of neuraminic acid; FIG. 3b).

### EXAMPLE 4

### Inhibition of GD3 Synthesis Protects from Stroke Damage

Shortly after inducing a transient focal ischemia as described in Reference Example 1, the animals were treated with the glucosylceramide synthase inhibitor D-threo-1-phenyl-2-decanoylamino-3-morpholino-1-propanol (D-PDMP). A single i.p. injection of D-PDMP (100 mg/kg of bodyweight; Biological Technology, London, GB) together with piperonyl butoxide (400 mg/kg of bodyweight; Fluka, Deisenhofen, Germany) was administered 15 min after occlusion of the MCA. Determination of GD3 by thin-layer chromatography revealed only a 10% increase in GD3 on the ischemic side compared with the non-ischemic side in these animals, whereas a 250% increase was detected in untreated animals (FIG. 4a).

Determination of the infarct volume as described in Reference Example 2 revealed an ischemic lesion with an average volume of 6.01±2.10 mm³ compared with 77.5±9.2 mm³ in untreated mice (n=5; FIG. 4b). Thus, inhibition of ganglioside neosynthesis in vivo resulted in a 90% reduction in the lesion caused by stroke compared with untreated ischemic animals (p <0.007). The map of the frequency distribution shows strong involvement of the hippocampus and hypothalamus in the infarct, while the other regions appear to be only slightly involved. The total amount of GD3 in the brains of animals treated with D-PDMP was 355% lower than in the brains of untreated ischemic animals.

We claim:

1. The use of at least one GD3 synthase inhibitor for the preparation of a pharmaceutical composition for the treatment of neuropathological disorders and signs, symptoms and dysfunctions associated therewith.

2. The use as claimed in claim 1 for treating cerebral ischemia.

3. The use as claimed in claim 2 for treating stroke, preferably acute stroke.

4. The use as claimed in claim 1 for treating traumatic damage to the brain and spinal cord.

5. The use as claimed in claim 1 for treating neurodegenerative disorders.

6. The use as claimed in claim 5 for treating dementia, especially Alzheimer's dementia, parkinsonism, ALS, multiple sclerosis.

7. A process for the preparation of a pharmaceutical composition for the treatment of neuropathological disorders, wherein at first a GD3 synthase inhibitor is selected from a variety of substances and this inhibitor is then used in the preparation of the pharmaceutical composition.
